(19) United States
(12) Patent Application Publication
(10) Pub. No.: US 2004/0213851 A1
(43) Pub. Date: Oct. 28, 2004

(54) Disinfecting polymer and articles made therefrom

(76) Inventor: Ihab M. Hekal, Greenwich, CT (US)

Correspondence Address: William H. Holt, 12311 Harbor Drive, Woodbridge, VA, 22192, US

(21) Appl. No.: 10/831,290

(22) Filed: Apr. 26, 2004

Related U.S. Application Data
(60) Provisional application No. 60/465,209, filed on Apr. 25, 2003.

(51) Int. Cl. A61K 9/14; A61K 33/14

(52) U.S. Cl. 424/486; 523/122

(57) ABSTRACT

A disinfecting polymer composition is created for emitting chlorine dioxide for use in precluding microbial growth. Products containing the polymer can be used in the form of film, sheets, or articles extruded or molded from the polymer for protecting against contamination of foods, leather goods in transit, or other products otherwise subject to contamination.

### BACKGROUND OF THE INVENTION

The use of chlorine dioxide to disinfect articles is well known. The reaction of alkaline or earth alkaline chlorite with organic or inorganic acids in the presence of water to produce chlorine dioxide is also well known.

The solid phase reaction of oxidizing inorganic compounds, with alkaline or earth alkaline chlorites, is also well known. The main problem is how can we embody any of the reaction in a form that can be safely manufactured in a form that can be used.

Solid acids when mixed with solid chlorites produce chlorine dioxide.

Lovely, U.S. Pat. No. 3,591,515 describes a chlorite-containing powder that release chlorine dioxide upon being admixed with an acid containing powder.

Hartshorn, U.S. Pat. No. 4,104,190 describes solid mixtures of sodium chlorite and citric, adipic or malic acid that are compressed to form tablets.

Mason et al., U.S. Pat. No. 4,547,381 and No. 4,689,169, disclose mixtures of powdered sodium chlorite, acid and inert inorganic diluent that release chlorine dioxide without exposing the mixtures to ambient moisture

Since most of the desired articles are in solid form, then the reaction has to take place in the solid state, between the solid chlorites and the solid acids. Solid chlorites are compounds, which should be handled very carefully since they can explode if compressed or impacted. Because it is a solid state reaction, and it takes place mainly on the surface, a large surface area is necessary to achieve a meaningful reaction. This means very fine powder particles. To obtain fine particles of alkaline or earth alkaline chlorite salts, in order to create a large surface area, scientists went through a lengthy procedure to avoid mechanical grinding. To get the reaction to start, an acid is needed. The acid can be finely ground or dissolved in organic solvent, or be part of polymer, and when they are mixed they can react, in the presence of moisture.

Wellinghoff et al. have formulated composites that include a hydrophobic phase containing an acid releasing agent and a hydrophilic phase containing chlorite or other anions. The composite is substantially free of water and gas (e.g., chlorine dioxide) until it is exposed to moisture. Once exposed to moisture, acid and hydronium ions are generated in the hydrophobic phase. The hydronium ions migrate to the hydrophilic phase and react with the anions to generate a gas such as chlorine dioxide from the composite. These composites are composed of and generate only substances used in foods or substances generally recognized as safe or inert substances. The composites can be used for food packaging and other applications where the substances can be ingested by or in contact with humans or animals. These composites are described in U.S. Pat. Nos. 5,360,609, 5,631,300, 5,639,295 and 5,650,446 and U.S. patent application Ser. Nos. 08/858,860, 08/858,859, 08/465,086, 08/461,716, and 08/461,304. U.S. Pat. No. 5,360,609 describes a hydrogen-bonded phase containing a dissolved chlorite salt and describes an amine as a polymerization component of the hydrogen-bonded phase. The amine and chlorite salt do not react to form an ammonium chlorite. Instead, the chlorite salt dissociates into chlorite anions and counter ions, and the amine remains intact.

Japanese Kokai Nos. 63/296,758, 63/274,434, and 57/168,977 describe deodorants containing chlorine dioxide incorporated in a polymer, ceramic beads, or calcium silicate wrapped in non-woven cloth, respectively.

Gels, which generate chlorine dioxide, for use as topical applications for disaffection are disclosed by Kenyon et al., Am. J. Vet. Res., 45(5), 1101 (1986). Chlorine-dioxide generating gels are generally formed, by mixing a gel containing suspended sodium chlorite, with a gel containing lactic acid, immediately prior to use, to avoid premature chlorine dioxide release.

Chlorine dioxide releasing gels have also been used in food preservation.

Tice et al., U.S. Pat. No. 4,585,482 describe gradual hydrolysis of alternating polyvinyl methyl ether maleic anhydride or polylactic glycolic acid to generate acid which can release chlorine dioxide from sodium chlorite. A polyalcohol humectant and water are encapsulated with the polyanhydride or polyacid in a nylon coating. After sodium chlorite is diffused into the capsule through the nylon wall, an impermeable polystyrene layer is coarsened around the nylon capsule. Solvents are required for reaction and application of the capsules. The capsules can be coated onto surfaces to release chlorine dioxide. Although the capsules are said to provide biocide action for several days to months, chlorine dioxide release begins immediately after the capsules are prepared. The batch wise process used to prepare the capsules also involves numerous chemical reactions and physical processes, some of which involve environmental disposal problems.

### SUMMARY OF THE INVENTION

A dispersion of fine particles of alkaline or earth-alkaline chlorite powder, in an inert polymer, that has an moisture vapor transmission rate (MVTR) from 0.1 to 100 gram, then added to that, a stoichiometric ratio of fine inorganic acid powder is produced and extruded it into film, sheet, fine beads, or molded into various articles which are then used for precluding or minimizing growth of microorganisms, spores and other toxins.

An important part of the invention is to provide a safe mode for producing the compound in a form that can be safely manufactured and used.

### BRIEF DESCRIPTION OF DRAWINGS

FIG. 1 shows an embodiment of the invention and illustrates a fragmentary sectional view of a threaded end closure, or cap, for a threaded container, or jar, and a ring polymeric ring containing chlorine dioxide and located in a threaded area of the container and cap therefor.

FIG. 2 shows a fragmentary sectional view of a modified form of threaded end closure, or cap, for a threaded container, or jar, wherein chlorine dioxide is incorporated into the closure or cap.

### DETAILED DESCRIPTION OF THE INVENTION

The invention relates to a novel composition of material and to the production of articles therefrom which are used to prevent or minimize microbial growth, spores and other toxins. Typical articles of manufacture that will benefit from the invention are closures for container and/or films for packaging and/or storage of food products such as seafood, meat, fruits and vegetables particularly those having low acidity, i.e., a pH above 5, such as melons and cantaloupes, and the like, as well as foot pads for use in shower rooms at sports clubs and athletic dressing rooms, pads for use as liners for shoes to preclude the growth and/or transfer of athlete foot fungus,

A composition is processed from alkaline or earth alkaline chlorite salts which were ground in a jet mill to a 5 microns mean particle size, then fed in a twin screw extruder with a low density polyethylene (V-LDPE), Dupont exact # 4023. The extruder temperature was below eighty (80) degrees Celsius. Sodium dihydrogen phosphate of mean particle size 5 microns, was fed into a second port of the extruder. The molten mixture was blown into 12 micron thin film.

A trace amount of moisture or anhydride is needed to start the reaction. Once the reaction starts, water is released, and that water sustains the reaction.

2NaH₂PO₄+5NaClO₂→2Na₃PO₄+4CLO₂+NaCl+2H₂O

In that reaction 5Cl+++→4CL+++++Cl−(Disproportionalization).

To avoid a spontaneous reaction and to be able to control the reaction, fine dry chlorites powder is dispersed in a molten, inert polymer such as polyethylene (PE) or polypropylene (PP), etc. with minimum agglomeration. Fine dry acid powder is added to that molten mixture and dispersed. During the dispersion process many of the acid powder particles come in contact with the chlorite powder particles. The mixture is either cooled and stored as pellets in absence of moisture for further processing, or cast into film or sheet or blown into film or injection molded into articles. The number of the chlorite particles in a volume unit of the mixture, determines the distance between the particles, and increases the probability of having a chlorite particle in contact with an acid particle. For that reason the rate of reaction is a function of concentration and the powder particle size. The diffusion constant and the solubility of water in polymer are important factors in establishing the rate of reaction. The more permeable the polymer to water and chlorine dioxide the faster the reaction. The concentration of the chlorite and its stoichiometric ratio of acid, determine the amount of produced ClO₂.

The foregoing disinfecting polymer can be formed into film, sheets, beads and articles such as wrappings and packaging, including bags and cartons, for seafood, meats, vegetables and fruits such as low acidity (pH above 5) melons, cantaloupes and the like, and sterile or semi-sterile packaging for tooth brushes, tampons and the like, as well as being adaptable in the manufacture of floor pads for shower and locker rooms and shoe liners for protecting against athletes foot fungus, in addition to packaging of, or with, leather ware to protect against microbial growth during shipment in humid conditions.

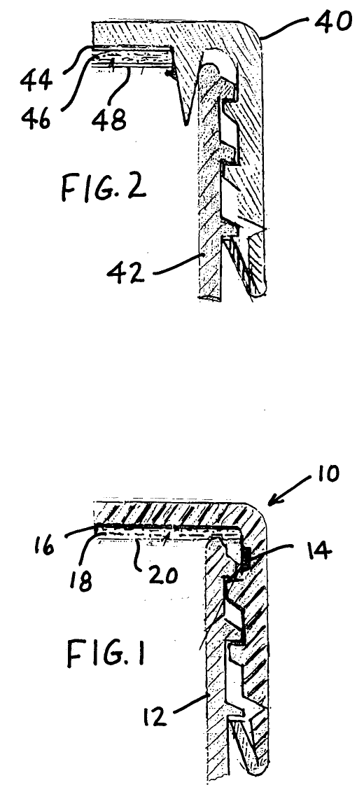

The disinfecting polymer can be used for protecting food products as described in the foregoing paragraph, and also for use in the manufacture of lids or closures for containers. As is shown in FIG. 1, one embodiment of the invention is illustrated in the form of a fragmentary sectional view of a threaded end closure, or cap, generally indicated by the numeral 10 which is used to close a threaded container, or jar, 12, the disinfecting dispersion being used in the form of an annular polymeric ring 14, containing the chlorine dioxide polymer and located in a threaded area of the container and cap therefor. The end closure or cap 10 is provided with an oxygen barrier in the form of a disk or insert 16, an underlying thin polypropylene layer 18 and an elastomer layer 20 including an oxygen absorber. In the absence of the present invention, food particles and the like which may become trapped in the threads during filling of the container 12 is not hermitically sealed and may become contaminated with spores or other toxins which then could contaminate the product upon opening of the container 12. The present invention emits chlorine dioxide from annular ring 14 which precludes microbial growth in any food trapped within the threaded area.

FIG. 2 illustrates a modified form of container and closure wherein a threaded end closure or lid 40 is threaded upon ajar or container 42. Lid 40 is manufactured a polymer or plastic which contains the disinfecting polymer of the present invention and which emits chlorine dioxide, particularly throughout the threaded area along the lid 40 and container 42. The closure or lid 40 is provided with an oxygen barrier 44, a layer 46 of an elastomer and oxygen absorber, and a polyolefin elastomer layer 48.

I claim:

1. A disinfecting polymer comprising:
first fine particles formed of alkaline or earth-alkaline chlorite,
second fine particles formed of inorganic acid, said second fine particles being contained at a stoichiometric amount relative to the first fine particles, and
a polymer containing the first fine particles and the second fine particles, said polymer having a moisture vapor transmission rate ranging from 0.1 to 100 gram.

2. A disinfecting polymer according to claim 1, wherein said first fine particles and said second fine particles are dispersed in the polymer at 10 to 100 wt. % of the polymer, respectively.

3. A disinfecting polymer according to claim 1, wherein said first fine particles and said second fine particles have an average diameter ranging from 0.1 to 100 micrometers, preferably from 0.1 to 20 micrometers, respectively.

4. A disinfecting polymer according to claim 1, wherein said alkaline or earth-alkaline chlorite is one selected from the group consisting of sodium, potassium, lithium, magnesium, calcium, and barium chlorite

5. A disinfecting polymer according to claim 1, wherein said inorganic acid is one selected from the group consisting of mono sodium or potassium phosphate, sodium or potassium hydrogen sulfate, AlCl₃, NaBF₄, and P₂O₅.

6. A disinfecting polymer according to claim 1, wherein said polymer includes at least one of polyethylene, polypropylene, polyolefins, polystyrene, synthetic rubbers, and vinyls,

8. A disinfecting polymer according to claim 1, wherein said first and second fine particles are dispersed in the polymer in a single or twin-screw extruder.

9. A disinfecting polymer according to claim 1, wherein said first and second fine particles are dispersed in the polymer in one of a single and twin-screw extruder with at least one feeding port.

10. A film or sheet formed of the disinfecting polymer according to claim 1.

11. A film or sheet according to claim 10, further comprising at least one layer of multi-layer film or sheet co-extruded or laminated on the film.

12. A film or sheet according to claim 10, further comprising at least one permeable layer and at least one barrier polymer layer co-extruded or laminated on the film.

13. A film or sheet according to claim 10, wherein said first and second fine particles produce chlorine dioxide through a reaction between the acid and the chlorite when activated with water.

14. A film or sheet according to claim 10, wherein said film is formed in a sanitary or diaper bag for waste disposal in a home or a hospital for stopping microbial growth to prevent bad odor.

15. A film or sheet according to claim 10, wherein said film is formed in an article to be packaged with leather ware.

16. A film or sheet according to claim 10, wherein said film is formed into a bag for packing fresh fish and sea food, chicken, meat to minimize spoiling during transportation from store to home.

17. A film or sheet according to claim 10, wherein said film is formed into a bag for packing sterile or semi sterile product (tampons, tooth brushes).

18. A film or sheet according to claim 10, wherein said film is formed in a bag for packing fresh fruits, and vegetables, particularly those have low acidity (pH above 5) such as melons and cantaloupes, etc.

19. A film or sheet according to claim 10, wherein said film is formed in a floor pad for use in locker rooms and shower rooms in a sport club and as a shoe cushion for preventing athletes foot.

20. An end closure including an annular ring comprised of the disinfecting polymer according to claim 1 wherein said annular ring is inserted between said end closure and a related container for protecting contained product from microorganisms.

21. An end closure comprised of the disinfecting polymer according to claim 1 wherein said end closure is provided with a liner comprised of an oxygen barrier for protecting product in a related container against emissions from said end closure.
